(19) United States
(12) Patent Application Publication
(10) Pub. No.: US 2019/0247508 A1
(43) Pub. Date: Aug. 15, 2019

(54) EXCIPIENT SYSTEM FOR TOPICAL DELIVERY OF PHARMACEUTICAL AGENTS

(71) Applicants: Turner Rockhill, Akron, OH (US); William H. Beeson, Carmel, IN (US)

(72) Inventors: Turner Rockhill, Akron, OH (US); William H. Beeson, Carmel, IN (US)

(73) Assignee: Ad Lunam Labs, Inc., Moorcroft, WY (US)

(21) Appl. No.: 16/392,830

(22) Filed: Apr. 24, 2019

Related U.S. Application Data
(63) Continuation of application No. 14/022,403, filed on Sep. 10, 2013.
(60) Provisional application No. 61/698,898, filed on Sep. 10, 2012.

(51) Int. Cl. A61K 47/46; A61K 36/185; A61K 38/18; A61K 31/506; A61K 8/92; A61Q 19/08; A61K 8/365; A61K 31/203; A61K 31/07; A61K 36/53

(52) U.S. Cl. CPC A61K 47/46; A61K 36/185; A61K 38/18; A61K 31/506; A61K 8/922; A61K 47/14; A61K 8/365; A61K 31/203; A61K 31/07; A61K 36/53; A61Q 19/08

(57) ABSTRACT

The subject invention is based upon the discovery that a wide variety of pharmaceutical agents can be delivered into the skin, fingernails, and toenails of patients by dissolving or dispersing the pharmaceutical agent in a solvent system which is comprised of a combination of an alkyl lactate and Simmondsia chinesis seed oil. The subject invention more specifically discloses a pharmaceutical serum which is comprised of (1) an alkyl lactate, wherein the alkyl group in the alkyl lactate contains from 2 to about 12 carbon atoms, (2) Simmondsia chinesis seed oil, and (3) a pharmaceutical agent. Some representative examples of pharmaceutical agent which can be incorporated into the pharmaceutical serums of this invention include, hormones, growth factors (cytokines), antimicrobials, antibacterials, antibiotics, non-steroidal anti-inflammatory agents, immunodilators, anesthetics, plant extracts, vitamins, corticosteroids, hair growth stimulants, and the like.

This application is a continuation application of U.S. patent application Ser. No. 14/022,403, filed on Sep. 10, 2013, which claims the benefit of U.S. Provisional Patent Application Ser. No. 61/698,898 filed on Sep. 10, 2012. The teachings of U.S. patent application Ser. No. 14/022,403 and U.S. Provisional Patent Application Ser. No. 61/698,898 are incorporated herein by reference in their entirety.

### BACKGROUND OF THE INVENTION

The effectiveness of virtually all pharmaceutical compositions which are applied to the skin of a patient is normally contingent upon delivery of the active ingredients of such composition through the stratum corneum and viable epidermis into the dermis layer of the skin structure. This is because the active ingredients in such compositions cannot be effective unless they penetrate through the dead layers of skin tissue and into the dermis layer of living skin cells. This is normally a difficult proposition for water soluble active ingredients, such as ascorbic acid, because the stratum corneum is a good water barrier. The stratum corneum and viable epidermis act to protect the body by holding water therein to prevent dehydration and by keeping external water which is frequently contaminated with harmful microorganisms and toxic chemicals out of the body. By the same token, the skin is also a good barrier with respect to most organic solvents. Accordingly, only a few solvents are capable of topically delivering pharmaceutical agents deeply into the skin.

Known solvents which are capable of penetrating through the stratum corneum and viable epidermis into the dermis layer of the skin structure cannot be beneficially used in many cases. This is because many pharmaceutical agents are not soluble in such solvents making effective topical delivery using them impossible. In other cases, the solvent may by chemically or biologically incompatible with pharmaceutical agent by virtue of reacting with it or otherwise destroying its bioavailability. In still other cases, human or animal exposure to the solvent may to detrimental due to toxicity and/or undesirable side effects. For instance, the long term exposure of humans or animals to solvents, such as dimethylsulfoxide, would not be desirable and its use on a repeated basis would not normally be warranted.

In many cases, by the topical administration of beneficial pharmaceutical agents to treat maladies in human patients and animals or to attain other desired beneficial results is simply not possible because a solvent system for the effective delivery of the pharmaceutical agent is simply not known. In other cases, pharmaceutical agents can be delivered topically to attain beneficial results. However, the results attained could be dramatically improved if better topical delivery systems were available. For instance, hair growth stimulants, such as minoxidil, can be topically delivered with some degree of success. However, the level of beneficial hair growth attained could be significantly improved if a delivery system to transport the hair growth stimulant deeper into the skin structure at a higher concentration was available.

There has been a long felt need for an excipient system which is capable of delivering a wide range of pharmaceutical compositions through the stratum corneum and viable epidermis into the dermis layer of the skin structure at high levels of concentration. Such an excipient system should be chemically and biologically inert with respect to the pharmaceutical agent. It should also be nontoxic and should not induce any undesirable side effects in the human patient or animal being treated. It is also of utmost importance for the excipient system to be a good solvent for the pharmaceutical agent or for the pharmaceutical agent to be highly dispersible in it.

### SUMMARY OF THE INVENTION

The subject invention is based upon the discovery that a wide variety of pharmaceutical agents can be delivered into the skin, fingernails, and toenails of patients by dissolving or dispersing the pharmaceutical agent in a solvent system which is comprised of a combination of an alkyl lactate and Simmondsia chinesis seed oil. It is critical for the solvent system to contain both the alkyl lactate and Simmondsia chinesis seed oil to attain penetration through skin, fingernails and toenails. In other words, for effective delivery of the pharmaceutical agent through the skin or nail it is critical for the pharmaceutical agent to be dissolved or dispersed in a mixture of an alkyl lactate and Simmondsia chinesis seed oil. The subject invention more specifically discloses a pharmaceutical serum which is comprised of (1) an alkyl lactate, wherein the alkyl group in the alkyl lactate contains from 2 to about 12 carbon atoms, (2) Simmondsia chinesis seed oil, and (3) a pharmaceutical agent. Some representative examples of pharmaceutical agent which can be incorporated into the pharmaceutical serums of this invention include, hormones, growth factors (cytokines), antimicrobials, antibacterials, antibiotics, non-steroidal anti-inflammatory agents, immunodilators, anesthetics, plant extracts, vitamins, corticosteroids, hair growth stimulants, and the like.

The subject invention more specifically discloses a pharmaceutical serum which is comprised of (1) an alkyl lactate, wherein the alkyl group in the alkyl lactate contains from 2 to about 12 carbon atoms, (2) Simmondsia chinesis seed oil, and (3) a pharmaceutical agent.

The subject invention further discloses a pharmaceutical serum which is comprised of (1) an alkyl lactate, wherein the alkyl group in the alkyl lactate contains from 2 to about 12 carbon atoms, (2) Simmondsia chinesis seed oil, and (3) a pharmaceutical agent, wherein the pharmaceutical agent is selected from the group consisting of hormones, growth factors, antimicrobials, antibacterials, antibiotics, non-steroidal anti-inflammatory agents, immunodilators, anesthetics, plant extracts, vitamins, corticosteroids, hair growth stimulants, and the like.

In one embodiment of this invention a hair growth stimulant is included in the pharmaceutical serum of this invention as the pharmaceutical agent. In most cases the hair growth stimulant will be a antihypertensive vasodilator medication, such as minoxidil (6-(1-piperidinyl)-2,4-pyrimidinediamine 3-oxide) which is included in the serum at a level which is within the range of about 0.5 weight percent to about 12 weight percent. The hair growth stimulant will more typically be included in the serum at a level which is within the range of about 1 weight percent to about 8 weight percent. The hair growth stimulant will most typically be included in the serum at a level which is within the range of about 2 weight percent to about 5 weight percent.

The present invention also reveals a method for treating human hair loss which comprised topically applying a hair growth serum to an area of skin where hair growth is desired, wherein the hair growth serum is comprised of (1) an alkyl lactate, wherein the alkyl group in the alkyl lactate contains from 2 to about 12 carbon atoms, (2) Simmondsia chinesis seed oil, and (3) from about 0.5 weight percent to about 12 weight percent of a hair growth stimulant.

In another embodiment of this invention an antioxidant is included in the pharmaceutical serum of this invention as the pharmaceutical agent. In most cases the antioxidant, such as carnosic acid, will be included in the pharmaceutical serum at a level which is within the range of about 0.01 weight percent to about 15 weight percent. The antioxidant will more typically be included in the serum at a level which is within the range of about 0.1 weight percent to about 12 weight percent. The antioxidant will most typically be included in the serum at a level which is within the range of about 0.5 weight percent weight percent to about 10 weight percent.

In another embodiment of this invention (2E,4E,6E,8E)-3,7-dimethyl-9-(2,6,6-trimethylcyclohex-1-enyl)nona-2,4,6,8-tetraen-1-ol, commonly known as retinol, or retinoic acid, commonly known as Retin-A, is included in the pharmaceutical serum of this invention as the pharmaceutical agent. Retinol and Retin-A are known to increase the production of cells in the top layer of skin, helping to rejuvenate the skin. As a result, the skin gradually looks younger in appearance. Retinol and Retin-A also induce higher levels of collagen production which makes to skin firmer and accordingly gives it a younger appearance. These agents may also reduce pigmentation issues that stem from sun damage. In any case retinol and Retin-A are known to help improve the overall appearance of the skin and to reduce the visible signs of aging. These agents are also highly effective antioxidants that can help prevent harmful carcinogens from breaking down skin cells. They also stimulate the production of healthy skin cells, which is essential in healing skin damage caused by aging and environmental exposure. Retinol and Retin-A also stimulates collagen production, which can help fill in the fine lines and wrinkles caused by age, shrink pores, and soften the skin.

By virtue of their ability to unclog pores and remove dead skin cells retinol and Retin-A are often used in treating patients suffering with acne. The healing properties of retinol can also help to reverse sun damage and relieve sunburn. Retinol and Retin-A are both derivatives of vitamin A and are sometimes used interchangeably due to confusion. However, Retin-A is much stronger and has a much more aggressive effect on the skin. As a consequence of Retin-A being significantly harsher and causing a higher incidence of side effects, such as skin redness, itchiness and rashes, retinol is much better for sensitive skin.

In most cases the retinol will be included in the pharmaceutical serum at a level which is within the range of about 0.1 weight percent to about 15 weight percent. On the other hand, retinoic acid will typically be included in such pharmaceutical serums at levels which are in the range of about 0.005 weight percent to about 0.5 weight percent. In any case, this invention further discloses a pharmaceutical serum which is comprised of (1) an alkyl lactate, wherein the alkyl group in the alkyl lactate contains from 2 to about 12 carbon atoms, (2) Simmondsia chinesis seed oil, and (3) a pharmaceutical agent which is selected from the group consisting of retinol and retinoic acid.

The present invention also reveals a method for skin rejuvenation which comprised topically applying a skin rejuvenation serum to an area of skin where skin rejuvenation is desired, wherein the skin rejuvenation serum is comprised of (1) an alkyl lactate, wherein the alkyl group in the alkyl lactate contains from 2 to about 12 carbon atoms, (2) Simmondsia chinesis seed oil, and (3) an effective amount of a vitamin A derivative selected from the group consisting of retinol and retinoic acid.

### DETAILED DESCRIPTION OF THE INVENTION

The pharmaceutical serum of this invention is comprised of (1) an alkyl lactate, (2) Simmondsia chinesis seed oil, and (3) a pharmaceutical agent. The alkyl lactate utilized will typically have an alkyl group that contains from 2 to about 12 carbon atoms and will accordingly be of the structural formula:

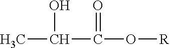

wherein R represents an straight chained or a branched alkyl group that contains from 2 to 12 carbon atoms. The alkyl group (R) of the alkyl lactate will typically contain from 2 to about 8 carbon atoms and will more typically contain from 3 to 6 carbon atoms. In many cases the alkyl group of the alkyl lactate will contain from 4 to 6 carbon atoms. For instance, the alkyl group of the alkyl lactate can contain 4, 5, or 6 carbon atoms. Some representative examples of alkyl lactates that can be used include: ethyl lactate, n-propyl lactate, iso-propyl lactate, n-butyl lactate, iso-butyl lactate, t-butyl lactate, isoamyl lactate, n-pentyl lactate, t-pentyl lactate, n-hexyl lactate, iso-hexyl lactate, t-hexyl lactate, n-heptyl lactate, iso-heptyl lactate, t-heptyl lactate, n-octyl lactate, iso-octyl lactate, and t-octyl lactate.

Isoamyl lactate is preferred for utilization as the alkyl lactate because it is not a volatile as the lower molecular weight alkyl lactates, but is still a low viscosity liquid at room temperature. Isoamyl lactate is of the structural formula:

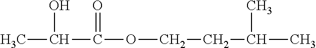

and is also a colorless liquid having a pleasant mild odor. Isoamyl lactate is preferred for utilization in conjunction with oil soluble antifungal agents, such as undecylenic acid. Mixtures of various alkyl lactates can be utilized in the pharmaceutical serum of this invention. For instance, a mixture of ethyl lactate and isoamyl lactate can be employed. Ethyl lactate is a colorless liquid of the structural formula:

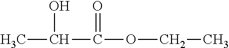

which is preferred for utilization in conjunction with antifungal agents which are water soluble. However, ethyl lactate has a strong odor. Accordingly, it is preferred to utilize ethyl lactate in mixtures with a higher molecular weight alkyl lactate to reduce volatility and the level of odor. It is desirable to utilize ethyl lactate in mixtures with isoamyl lactate in some cases. The weight ratio of ethyl lactate to isoamyl lactate will typically be within the range of about 1:10 to about 20:1. The weight ratio of ethyl lactate to isoamyl lactate will more typically be within the range of about 1:5 to about 10:1. Such mixtures of ethyl lactate and isoamyl lactate will preferably contain from about 30 weight percent to 70 weight percent ethyl lactate and from about 30 weight percent to about 70 weight percent isoamyl lactate. Such mixtures of ethyl lactate and isoamyl lactate will more preferably contain from about 40 weight percent to 60 weight percent ethyl lactate and from about 40 weight percent to about 60 weight percent isoamyl lactate. Such mixtures of ethyl lactate and isoamyl lactate will most preferably contain from about 45 weight percent to 55 weight percent ethyl lactate and from about 45 weight percent to about 55 weight percent isoamyl lactate.

The Simmondsia chinesis seed oil used in the antifungal serums of this invention is commonly known as jojoba oil or goat-nut oil. The Simmondsia chinesis seed oil can be expeller processed or it can be cold pressed at a temperature which does not exceed 150° F. (66° C.) and which preferably does not exceed 120° F. (49° C.). The Simmondsia chinesis seed oil is preferably golden Simmondsia chinesis seed oil which is filtered, but which is not refined. Accordingly, the Simmondsia chinesis seed oil will normally be filtered to remove undesired particulate matter. In some cases refined Simmondsia chinesis seed oil can by used in the pharmaceutical serums of this invention with good results. Such refined Simmondsia chinesis seed oil is clear, rather than being golden in color, and offers the advantage of having an extended shelf-life without becoming rancid. However, in the practice of the subject invention color is not of importance and oil stabilization can be achieved by adding a small amount of a soluble antioxidant, such as Vitamin E to the antifungal serum. In any case, the Simmondsia chinesis seed oil can be refined by extracting it from unrefined material with an organic solvent, such as n-hexane or cyclohexane, and then fractionally distilling the extract to remove the organic solvent.

In cases where Vitamin E (α-tocopherol, β-tocopherol, γ-tocopherol, and/or δ-tocopherol) is utilized in the pharmaceutical serum it is typically present at a level which is within the range of about 0.01 weight percent to about 2 weight percent, based upon the total weight of the antifungal serum. In cases where Vitamin E is utilized in the pharmaceutical serum it is more typically added at a level which is within the range of 0.05 weight percent to about 1 weight percent, and is generally employed at a level which is within the range of 0.1 weight percent to 0.5 weight percent, based upon the total weight of the pharmaceutical serum.

A wide variety of pharmaceutical agents can be utilized in the pharmaceutical serums of this invention. For instance, the pharmaceutical agent can be selected from hormones, growth factors, antimicrobials, antibacterials, antibiotics, non-steroidal anti-inflammatory agents, immunodilators, anesthetics, plant extracts, vitamins, vitamin derivatives, corticosteroids, hair growth stimulants, and the like.

In one embodiment of this invention the pharmaceutical agent is carnosic acid which is a naturally occurring antioxidant. Carnosic acid can be included in the formulations of this invention to provide a serum which can be topically applied to skin to provide it with a higher level of protection against photo-induced and other types of oxidative attack. The carnosic acid will typically be included in the skin cream formulation at a level which is within the range of 0.01 weight percent to 3 weight percent. It is normally preferred to include the carnosic acid at a level which is within the range of 0.05 weight percent to 1.5 weight percent with levels of 0.1 weight percent to 1 weight percent being most preferred.

The carnosic acid is naturally found in Libiatae plants, such as rosemary, marjoram, and sage. U.S. Pat. Nos. 5,859,293 and 5,256,700 disclose techniques for extracting high purity carnosic acid from rosemary and sage. For example, U.S. Pat. No. 5,256,700 discloses a process for obtaining carnosic acid comprising extracting a vegetable material selected from the group consisting of sage and rosemary with an apolar solvent to obtain an extract containing apolar compounds including carnosic acid, contacting the extract with an adsorbent material having an affinity for polar compounds for adsorbing the carnosic acid to separate the carnosic acid from the apolar compounds of the extract, desorbing the adsorbent material with a polar solvent to obtain the carnosic acid in the solvent and then evaporating the polar solvent from the carnosic acid to obtain a residue containing the carnosic acid.

Some methods for the preparation of carnosic acid by chemical synthesis have also been proposed in the literature by W. L. Meyer et al. [Tetrahedron Letters 1966, 4261; 1968, 2963; J. Org. Chem. 41, 1005 (1976)]. However, the syntheses involved are long and complex and, for economic reasons, cannot be applied to an industrial process. In addition, these syntheses lead to racemic mixtures of carnosic acid precursors and not to the pure enantiomers. It should also be pointed out that these works stop at the preparation of carnosic acid precursors and omit to describe the final preparation step(s). Another method of obtaining carnosic acid has been described in the literature by Brieskorn and Domling [Arch. Pharm. 302, 641 (1969)], comprising the catalytic reduction of carnosol. Once again, the application of this process on a large scale is not be envisaged because carnosol is not readily available on a commercial basis. For these reasons the carnosic acid used in the skin creams formulations of this invention will normally be obtained by extraction from a Libiatae plant, such as rosemary or marjoram. Accordingly, rosemary or marjoram extract will typically be used in the practice of this invention as the source of carnosic acid. However, to reduce the possibility of allergic reactions to the skin cream formulation the skin cream formulation will preferably be free of rosemary, sage, marjoram and other Libiatae plants.

Carnosic acid will typically be incorporated into the pharmaceutical serums of this invention as the extract of a Libiatae plant, such as rosemary or marjoram. In most cases the extract of the Libiatae plant will be included in the pharmaceutical serum at a level of about 0.5 weight percent to about 30 weight percent. The extract of the Libiatae plant will typically be included in the pharmaceutical serum at a level of about 1 weight percent to about 15 weight percent, and will more typically be included at a level of about 2 weight percent to about 10 weight percent. It is normally preferred to include the extract of the Libiatae plant in the pharmaceutical serum at a level of about 3 weight percent to about 8 weight percent. Such a sink rejuvenation serum can optionally contain up to about 5 weight percent glycerin and up to about 5 weight percent propylene glycol. It is typically preferred for such compositions to contain from about 0.05 weight percent to about 3 weight percent glycerin and from about 0.01 weight percent to about 3 weight percent propylene glycol. It is typically more preferred for such compositions to contain from about 0.1 weight percent to about 1 weight percent glycerin and from about 0.05 weight percent to about 0.5 weight percent propylene glycol.

In another embodiment of this invention idebenone is included in the pharmaceutical serums of this invention as an antioxidant. Idebenone will typically be present in such pharmaceutical serums at a level which is within the range of about 0.01 weight percent to about 5 weight percent. The idebenone will preferably be present in such pharmaceutical serums formulation at a level which is within the range of about 0.05 weight percent to about 3 weight percent and will more preferably be present at a level which is within the range of about 0.1 weight percent to about 1 weight percent.

Palmitoyl pentapeptide stimulates human fibroblasts to produce collagen and elastin which fight wrinkle formation and can reduce or eliminate existing wrinkles. However, as with all active ingredients in antiwrinkle formulations palmitoyl pentapeptide needs to be delivered deep into the dermis of the skin structure to attain a maximum level of effectiveness. In still another embodiment of this invention pharmaceutical serums can be compounded with palmitoyl pentapeptide to facilitate its topical delivery deep into the dermis of human patients. In such an embodiment of this invention the palmitoyl pentapeptide can accordingly be included in the pharmaceutical serum at a level which is within the range of about 0.05 weight percent to about 8 weight percent. In such pharmaceutical serums the palmitoyl pentapeptide will more typically be included at a level which is within the range of about 0.5 weight percent to about 6 weight percent, and will preferably be include at a level which is within the range of about 1 weight percent to about 5 weight percent. The palmitoyl pentapeptide will more preferably be included in the pharmaceutical serums of this invention at a level which is within the range of 2 weight percent to 4 weight percent.

In a further embodiment of this invention a hair growth stimulant is included in the pharmaceutical serum of this invention as the pharmaceutical agent. In most cases the hair growth stimulant will be a antihypertensive vasodilator medication, such as minoxidil (6-(1-piperidinyl)-2,4-pyrimidinedia mine 3-oxide) which is included in the serum at a level which is within the range of about 0.5 weight percent to about 12 weight percent. The hair growth stimulant will more typically be included in the serum at a level which is within the range of about 1 weight percent to about 8 weight percent. The hair growth stimulant will most typically be included in the serum at a level which is within the range of about 2 weight percent to about 5 weight percent. Such hair growth serums can be topically applied to an area of skin where hair growth is desired to facilitate such hair growth. In most cases the hair growth serum will be applied repeatedly to the area of skin over an extended period of time during which hair growth and hair maintenance in the area is desired. In most cases the hair growth serum will be applied at least once a day and can optionally be multiple times each day, such as twice a day. For instance, the hair growth serum can be applied every morning and in the evening during the treatment period which can extend over many years.

In another embodiment of this invention a vitamin A derivative selected from the group consisting of retinol and retinoic acid is included in a skin rejuvenation serum. Retinol based skin rejuvenation serums will typically contain retinol a level which is within the range of about 0.1 weight percent to about 15 weight percent. Such serums will more typically contain from about 0.5 weight percent to about 12 weight percent retinol and will more typically contain from 1 weight percent to 10 weight percent retinol. It is preferred for such skin rejuvenation serums to contain from about 3 weight percent to about 8 weight percent retinol. On the other hand, retinoic acid based skin rejuvenation serums will typically contain from about 0.005 weight percent to about 0.5 weight percent retinoic acid. Such skin rejuvenation serums will more typically contain from about 0.01 weight percent to about 0.4 weight percent retinoic acid and will preferably contain from about 0.02 weight percent to about 0.3 weight percent retinoic acid. Such skin rejuvenation serums will more preferably contain from about 0.03 weight percent to about 0.2 weight percent retinoic acid and will most preferably contain from about 0.04 weight percent to about 0.1 weight percent retinoic acid.

In a further embodiment of this invention an antimicrobial agent is included in the pharmaceutical serum of this invention. Some representative examples of antimicrobial agents that can be use include 2,4,4′-trichloro-2′-hydroxydiphenyl ether (or triclosan), 3,4,4′-trichlorobanilide, phenoxyethanol, phenoxypropanol, phenoxyisopropanol, hexamidine isethionate, metronidazole and its salts, miconazole and its salts, itraconazole, terconazole, econazole, ketoconazole, saperconazole, fluconazole, clotrimazole, butoconazole, oxiconazole, sulfaconazole, sulconazole, terbinafine, ciclopirox, ciclopiroxolamine, undecylenic acid and its salts, benzoyl peroxide, 3-hydroxybenzoic acid, 4-hydroxybenzoic acid, phytic acid, N-acetyl-L-cysteine acid, lipoic acid, azelaic acid and its salts, arachidonic acid, resorcinol, 2,4,4′-trichloro-2′-hydroxydiphenyl ether, 3,4,4′-trichlorocarbanilide, octopirox, octoxyglycerine, octanoylglycine, caprylyl glycol, 10-hydroxy-2-decanoic acid, dichlorophenyl imidazole dioxolane and its derivatives, described in patent WO 93/18743, farnesol and phytosphingosines, and mixtures thereof. The preferred antibacterial agents are triclosan, phenoxyethanol, octoxyglycerine, octanoylglycine, 10-hydroxy-2-decanoic acid, caprylyl glycol, farnesol and azelaic acid. Such antimicrobial agents will typically be included at a level which is within the range of 0.1 weight percent to 20 weight percent and will more typically be included at a level which is within the range of preferably from 0.2 weight percent to 10 weight percent relative to the total weight of the pharmaceutical serum.

In still another embodiment of this invention the pharmaceutical serum can include an agent for stimulating the synthesis of dermal or epidermal macromolecules and/or for preventing their degradation as the pharmaceutical agent. Among the active agents for stimulating dermal macromolecules or for preventing their degradation, mention may be made of those that act: either on collagen synthesis, such as extracts of Centella asiatica; asiaticosides and derivatives; ascorbic acid or vitamin C and its derivatives; synthetic peptides such as lamin, biopeptide CL or the palmitoyloligopeptide sold by the company Sederma; peptides extracted from plants, such as the soybean hydrolysate sold by the company Coletica under the trade name Phytokine®; and plant hormones such as auxins and lignans; or on elastin synthesis, such as the extract of Saccharomyces cerevisiae sold by the company LSN under the trade name Cytovitin®; and the extract of the alga Macrocystis pyrifera sold by the company Secma under the trade name Kelpadelie®; or on glycosaminoglycan synthesis, such as the product of fermentation of milk with Lactobacillus vulgaris, sold by the company Brooks under the trade name Biomin yogourth®; the extract of the brown alga Padina pavonica sold by the company Alban Muller under the trade name HSP3; and the extract of Saccharomyces cerevisiae available especially from the company Silab under the trade name Firmalift® or from the company LSN under the trade name Cytovitin®; or on fibronectin synthesis, such as the extract of the zooplankton Salina sold by the company Seporga under the trade name GP4G®; the yeast extract available especially from the company Alban Muller under the trade name Drieline®; and the palmitoyl pentapeptide sold by the company Sederma under the trade name Matrixil®; or on the inhibition of metalloproteases (MMPs), such as, more particularly, MMP 1, 2, 3 or 9. Mention may be made of: retinoids and derivatives, oligopeptides and lipopeptides, lipoamino acids, the malt extract sold by the company Coletica under the trade name Collalift®; extracts of blueberry or of rosemary; lycopene; isoflavones, their derivatives or plant extracts containing them, in particular extracts of soybean (sold, for example, by the company Ichimaru Pharcos under the trade name Flavosterone SB®), of red clover, of flax, of kakkon, or of sage; or on the inhibition of serine proteases such as leukocyte elastase or cathepsin G. Mention may be made of: the peptide extract of Leguminosa seeds (Pisum sativum) sold by the company LSN under the trade name Parelastyl®; heparinoids; and pseudodipeptides such as {2-[acetyl-(3-trifluoromethylphenyl)amino]-3-methylbutynylamino}acetic acid.

Among the active agents that stimulate epidermal macromolecules, such as fillagrin and keratins, mention may be made especially of the extract of lupin sold by the company Silab under the trade name Structurine®; the extract of beech Fagus sylvatica buds sold by the company Gattefosse under the trade name Gatuline®; and the extract of the zooplankton Salina sold by the company Seporga under the trade name GP4G®.

The pharmaceutical serum of this invention can optionally contain an agent for stimulating the proliferation of fibroblasts or keratinocytes and/or keratinocyte differentiation as the pharmaceutical agent. The agents for stimulating the proliferation of fibroblasts that may be used in the composition according to the invention may be chosen, for example, from plant proteins or polypeptides, extracts, especially of soybean (for example an extract of soybean sold by the company LSN under the name Eleseryl SH-VEG 8 or sold by the company Silab under the trade name Raffermine®); and plant hormones such as giberrellins and cytokinins.

The agents for stimulating the proliferation of keratinocytes that may be used in the composition according to the invention especially comprise retinoids, such as retinol and its esters, including retinyl palmitate; phloroglucinol; extracts of nut cakes sold by the company Gattefosse; and extracts of Solanum tuberosum sold by the company Sederma.

The agents for stimulating keratinocyte differentiation comprise, for example, minerals such as calcium; the extract of lupin sold by the company Silab under the trade name Photopreventine®; sodium beta-sitosteryl sulphate sold by the company Seporga under the trade name Phytocohesine®; and the extract of corn sold by the company Solabia under the trade name Phytovityl®; and lignans such as secoisolariciresinol. The composition according to the invention comprising these compounds is preferably intended to be used for preventing or treating signs of ageing of the skin.

The pharmaceutical serum of this invention can optionally further contain a dermo-decontracting agent as the pharmaceutical agent. The dermo-decontracting agents that may be used in the pharmaceutical serum of this invention include alverine and its salts, manganese gluconate, Diazepam, the hexapeptide argireline R sold by the company Lipotec, certain carbonylated secondary and tertiary amines, adenosine, and also sapogenins and the natural extracts, in particular of Wild Yam, containing them.

The pharmaceutical serum of this invention can optionally further contain agents for acting on the capillary circulation as their pharmaceutical agent. The active agents acting on the capillary circulation (vasoprotective or vasodilating agents) may be chosen from flavonoids, ruscogenins, esculosides, escin extracted from common horse chestnut, nicotinates, heperidine methyl chalcone, essential oils of lavender or of rosemary, and extracts of Ammi visnaga. The amount of these active agents may vary within a wide range. preferably from 0.05% to 10% by weight relative to the total weight of the pharmaceutical serum.

The pharmaceutical serum of this invention can optionally further contain agents acting on the energy metabolism of cells as their pharmaceutical serum. The active agents concerned are those acting on the energy metabolism of the skin, for instance, and in a non-limiting manner, ATP synthesis, and also those involved in the respiratory chain of the cell or in the energy reserves. Mention may be made of coenzyme Q10 (ubiquinone), cytochrome C, creatine or phosphocreatine.

The alkyl lactate will typically be present in the pharmaceutical serums of this invention at a level which is within the range of about 5 weight percent to about 80 weight percent and will more typically be present at a level which is within the range of about 10 weight percent to about 70 weight percent. The alkyl lactate will commonly be present in the pharmaceutical serum of this invention at a level which is within the range of about 10 weight percent to about 60 weight percent and will more commonly be present at a level which is within the range of about 15 weight percent to about 60 weight percent. In most cases, the alkyl lactate will typically be present in the pharmaceutical serum of this invention at a level which is within the range of about 15 weight percent to about 50 weight percent.

The Simmondsia chinesis seed oil will typically be present in the pharmaceutical serum of this invention at a level which is within the range of f about 5 weight percent to about 80 percent and more typically be present at a level which is within the range of about 10 weight percent to about 70 percent. The Simmondsia chinesis seed oil will commonly be present in the pharmaceutical serum of this invention at a level which is within the range of about 10 weight percent to about 60 percent and more typically will be present at a level which is within the range of about 15 weight percent to about 50 percent. In most cases, the Simmondsia chinesis seed oil will be present in the pharmaceutical serum of this invention at a level which is within the range of about 15 weight percent to about 45 percent and in many cases will preferably be present at a level which is within the range of about 15 weight percent to about 40 percent.

The level of pharmaceutical agent which is present in the pharmaceutical serums of this invention will very greatly with the nature of the pharmaceutical agent and the therapeutic result which is desired. However, the pharmaceutical agent will typically be present in the pharmaceutical serums of this invention at a level which is within the range of 0.001 weight percent to 40 weight percent and will more typically be present at a level which is within the range of about 0.01 weight percent to 35 weight percent. The pharmaceutical agent will commonly be present in the pharmaceutical serums of this invention at a level which is within the range of 0.02 weight percent to 30 weight percent and can be present at a level which is within the range of about 0.025 weight percent to 25 weight percent. For instance, the pharmaceutical agent can be present in the pharmaceutical serums of this invention at a level which is within the range of 0.05 weight percent to 25 weight percent. In some cases the pharmaceutical agent will be present at a relatively low level which is within the range of 0.01 weight percent to 0.5 weight percent or which is within the range of 0.02 weight percent to 0.3 weight percent. In other cases the pharmaceutical agent will be present at a relatively level which is within the range of 0.1 weight percent to 25 weight percent or which is within the range of 5 weight percent to 25 weight percent or which is within the range of 10 weight percent to 25 weight percent or which is within the range of 15 weight percent to 25 weight percent.

The pharmaceutical serum of this invention can also include a wide variety of other oils. These additional oils are typically vegetable oils (oils of plant origin), mineral oils (liquid petroleum jelly), oils of animal origin (such as lanolin), synthetic oils (perhydrosqualene), or a silicone oils (cyclomethicone). The vegetable oils that can be included in the pharmaceutical serum of this invention include avocado oil, soybean oil, coconut oil, shea butter, almond oil, eucalyptus essential oil, olive oil, hazelnut oil, walnut oil, peanut oil, corn oil, caster oil, soy oil, canola oil, rapeseed oil, cottonseed oil, palm oil, sesame oil, sunflower oil, safflower oil, rice bran oil, borage seed oil, syzigium aromaticum oil, hempseed oil, flaxseed oil, rape seed oil, evening primrose oil, rosehip oil, and melaleuca oil. Some representative examples of animal based oils that can optionally be utilized include lanolin, various fish oils, such as herring oil, cod-liver oil, and salmon oil. Typically, the pharmaceutical serum of this invention will be void of these additional oils since they are not believed to serve any beneficial purpose and dilute the levels of the more desirable components of the serum, such as the Simmondsia chinesis seed oil and the alkyl lactate which facilitate the delivery of the pharmaceutical agent into the skin. Accordingly, in cases where such oils are included in the pharmaceutical serum their total level will normally be limited to be within the range of 0 weight percent to about 25 weight percent and will more typically be limited to be within the range of 0 weight percent to 10 weight percent, based upon the total weight of the pharmaceutical serum. In cases where such additional oils are included they will normally be present in a total amount which is within the range of about 1 weight percent to about 5 weight percent.

Fatty alcohols (cetyl alcohol), fatty acids, petrolatum, and waxes (carnauba wax or ozokerite) can also optionally be included in the pharmaceutical serum of this invention. Petrolatum or mineral oil components, which when selected will generally be USP or NF grade. The petrolatum may be white or yellow. The viscosity or consistency grade of petrolatum is not narrowly critical. Petrolatum can be partially replaced with mixtures of hydrocarbon materials, which can be formulated to resemble petrolatum in appearance and consistency. For example, mixtures of petrolatum or mineral oil with different waxes and the like may be combined. Preferred waxes include bayberry wax, candelilla wax, ceresin, jojoba butter, lanolin wax, montan wax, ozokerite, polyglyceryl-3-beeswax, polyglyceryl-6-pentastearate, microcrystalline wax, paraffin wax, isoparaffin, vaseline solid paraffin, squalene, oligomer olefins, beeswax, synthetic candelilla wax, synthetic carnauba, synthetic beeswax and the like may be blended together. Typically, the pharmaceutical serum of this invention will be void of petrolatum and waxes since they are not believed to serve any beneficial purpose and dilute the levels of more desirable ingredients. Accordingly, in cases where petrolatum and/or waxes are included in the pharmaceutical serum their total level will normally be limited to be within the range of 0 weight percent to about 25 weight percent and will more typically be limited to be within the range of 0 weight percent to 10 weight percent, based upon the total weight of the pharmaceutical serum. In cases where petrolatum and/or such waxes are included they will normally be present in a total amount which is within the range of about 1 weight percent to about 5 weight percent.

The pharmaceutical serum of this invention can be made by simply mixing the desired constituents under conditions that are adequate to attain an essentially homogeneous mixture at a temperature which is typically within the range of about 10° C. to about 100° C. In most cases this can be accomplished by mixing the constituents at room temperature (18° C. to 23° C.). However, in some cases it is desirable to heat the components being mixed to a slightly elevated temperature which is within the range of about 30° C. to 60° C. to facilitate mixing.

This invention is illustrated by the following examples that are merely for the purpose of illustration and are not to be regarded as limiting the scope of the invention or the manner in which it can be practiced. Unless specifically indicated otherwise, parts and percentages are given by weight.

### EXAMPLE 1

In this experiment a skin rejuvenation serum of this invention was prepared by adding 90 ml of jojoba oil (filtered and unrefined), 5 ml of rosemary extract, and 5 ml of isoamyl lactate to a 150 ml beaker. Then, the mixture of liquids was then well mixed with a stirring rod to make a skin rejuvenation serum.

### EXAMPLE 2

In this experiment a skin rejuvenation serum of this invention was prepared by adding 88 ml of jojoba oil (filtered and unrefined), 5 ml of rosemary extract, 5 ml of isoamyl lactate, 1 ml of glycerin, and 1 ml of propylene glycol to a 150 ml beaker. Then, the mixture of liquids was then well mixed with a stirring rod to make a skin rejuvenation serum.

### EXAMPLE 3

In this experiment a skin rejuvenation serum of this invention was prepared by adding about 50 ml of jojoba oil (filtered and unrefined), 5 ml of rosemary extract, and 50 ml of isoamyl lactate to a 150 ml beaker. Then, the mixture of liquids was well mixed with a stirring rod to make a skin rejuvenation serum.

After about one week of treatment the black color of the fungus lightened and the toenail returned to a relatively normal appearance. The application of the antifungal serum was continued in the morning and in the evening for about 6 months to allow the infected toenail to completely grow out leaving only a normal nail structure. There was no reoccurrence of the onychomycosis after another 6 months. In other words, the patient remained free of fungus for 6 months after discontinuing treatment with the antifungal serum.

While certain representative embodiments and details have been shown for the purpose of illustrating the subject invention, it will be apparent to those skilled in this art that various changes and modifications can be made therein without departing from the scope of the subject invention.

What is claimed is:

1. A skin rejuvenation serum which is comprised of (1) an alkyl lactate, wherein the alkyl group in the alkyl lactate contains from 2 to about 12 carbon atoms, (2) Simmondsia chinesis seed oil, and (3) a vitamin A derivative selected from the group consisting of retinol and retinoic acid, wherein the alkyl lactate is present at a level which is within the range of about 5 weight percent to about 90 weight percent, and wherein the Simmondsia chinesis seed oil is present at a level of about 5 weight percent to about 90 percent.

2. The skin rejuvenation serum as specified in claim 1 wherein the vitamin A derivative is retinol.

3. The skin rejuvenation serum as specified in claim 2 wherein the retinol is present at a level which is within the range of about 0.1 weight percent to about 15 weight percent, based upon the total weight of the skin rejuvenation serum.

4. The skin rejuvenation serum as specified in claim 2 wherein the retinol is present at a level which is within the range of 1 weight percent to 10 weight percent retinol.

5. The skin rejuvenation serum as specified in claim 2 wherein the retinol is present at a level which is within the range of 3 weight percent to 8 weight percent retinol.

6. The pharmaceutical serum as specified in claim 3 wherein the alkyl lactate contains from 4 to 6 carbon atoms.

7. The pharmaceutical serum as specified in claim 4 wherein the alkyl lactate is isoamyl lactate.

8. The pharmaceutical serum as specified in claim 3 wherein the alkyl lactate is ethyl lactate.

9. A pharmaceutical serum as specified in claim 3 wherein the Simmondsia chinesis seed oil is golden Simmondsia chinesis seed oil.

10. A pharmaceutical serum as specified in claim 3 wherein the Simmondsia chinesis seed oil is filtered.

11. A pharmaceutical serum as specified in claim 3 wherein the Simmondsia chinesis seed oil is refined.

12. The skin rejuvenation serum as specified in claim 1 wherein the vitamin A derivative is retinoic acid.

13. The skin rejuvenation serum as specified in claim 12 wherein the retinoic acid is present at a level which is within the range of about 0.005 weight percent to about 0.5 weight percent, based upon the total weight of the skin rejuvenation serum.

14. The skin rejuvenation serum as specified in claim 12 wherein the retinoic acid is present at a level which is within the range of about 0.01 weight percent to about 0.4 weight percent.

15. The skin rejuvenation serum as specified in claim 12 wherein the retinoic acid is present at a level which is within the range of about 0.02 weight percent to about 0.3 weight percent.

16. The skin rejuvenation serum as specified in claim 12 wherein the retinoic acid is present at a level which is within the range about 0.03 weight percent to about 0.2 weight percent.

17. The skin rejuvenation serum as specified in claim 12 wherein the retinoic acid is present at a level which is within the range of about 0.04 weight percent to about 0.1 weight percent retinoic acid.

18. The pharmaceutical serum as specified in claim 12 wherein the alkyl lactate is isoamyl lactate.

19. The pharmaceutical serum as specified in claim 12 wherein the alkyl lactate is ethyl lactate.

20. A skin rejuvenation serum which is comprised of (1) an alkyl lactate, wherein the alkyl group in the alkyl lactate contains from 2 to about 12 carbon atoms, (2) Simmondsia chinesis seed oil, and (3) palmitoyl pentapeptide.
